(19) United States
(12) Patent Application Publication
(10) Pub. No.: US 2026/0137276 A1
(43) Pub. Date: May 21, 2026

(54) INTRAOCULAR PRESSURE TONOMETERS, METHODS OF FABRICATOIN AND SYSTEMS AND METHODS FOR MONITORING INTRAOCULAR PRESSURE

(71) Applicant: Purdue Research Foundation, West Lafayette, IN (US)

(72) Inventors: Chi Hwan Lee, West Lafayette, IN (US); Pedro Irazoqui, Baltimore, MD (US); Bryan Boudouris, West Lafayette, IN (US); Kyunghun Kim, Hwaseong-si (KR); Ho Joong KIm, West Lafayette, IN (US)

(21) Appl. No.: 18/705,896

(22) PCT Filed: Oct. 28, 2022
(86) PCT No.: PCT/US2022/048173

§ 371 Date: Apr. 29, 2024

Related U.S. Application Data
(60) Provisional application No. 63/273,377, filed on Oct. 29, 2021.

(51) Int. Cl. A61B 3/16; A61B 3/00; A61B 5/00

(52) U.S. Cl. CPC A61B 3/16; A61B 3/0025; A61B 5/6802; A61B 2560/0214; A61B 2562/12; A61B 2562/164

(57) ABSTRACT

Intraocular pressure (IOP) tonometers, systems including IOP tonometers, methods of fabricating IOP tonometers, and methods for using systems to monitor IOP in a subject. The IOP tonometers include a contact lens configured to be located and retained on a user's eye, and a corneal sensor having a circular trace located proximate an outer peripheral edge of the contact lens so as to leave an unobstructed area at a center region of the contact lens. The corneal sensor produces a detectable shift in resonance frequency in response to a change in IOP of the user's eye, which is wirelessly delectable by a receiving antenna. The IOP tonometer is fabricated by depositing one or more layers of the circular trace onto a temporary substrate and then transferring the circular trace onto the outer surface of the contact lens.

### CROSS-REFERENCE TO RELATED APPLICATIONS

This application claims the benefit of Provisional U.S. patent application Ser. No. 63/273,377, filed Oct. 29, 2021, the contents of which are incorporated by reference herein in their entirety.

### BACKGROUND OF THE INVENTION

The present invention generally relates to intraocular pressure (IOP) tonometers, systems including IOP tonometers, methods of fabricating IOP tonometers, and methods for monitoring IOP in subjects.

Glaucoma, which is typically caused by abnormal increases in intraocular pressure IOP, remains the second leading cause of blindness worldwide, and gradually steals vision without early warning signs or pain. IOP is commonly determined using various types of equipment referred to as tonometers that measure the fluid pressure inside the eye. Currently, the most effective defense against the progression of glaucoma is monitoring and lowering a patient's mean IOP. The largest peaks and fluctuations of IOP typically appear overnight during sleep, whereas daytime IOP tends to be lower than a threshold that is often associated with vision damage. Unfortunately, mean IOP measurements are usually obtained using tonometers during daytime office hours when medical providers are operating and therefore are unlikely to provide complete information. Additionally, frequent office visits are a significant burden for many patients living in low-income or rural communities.

To alleviate these issues, portable IOP tonometers have been used to support home-based continuous monitoring. However, existing tonometers are configured to obtain IOP recordings only when a patient is awake and only when the patient manually initiates the measurement process. Studies suggest that the diagnosis accuracy of glaucoma could increase by more than fifty percent if continuous 24-hour reading of IOP could be analyzed instead of relying on the current daytime in-office measurements. Yet, no known existing approaches are capable of accomplishing this goal.

In view of the above, it can be appreciated that there are certain problems, shortcomings or disadvantages associated with conventional techniques of obtaining IOP measurements, and that it would be desirable if devices, systems and methods were available for continuously monitoring IOP change in a subject that were capable of at least partly overcoming or mitigating one or more of the problems, shortcomings, or disadvantages noted above.

### BRIEF DESCRIPTION OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, devices, systems and methods adapted for continuously monitoring IOP change in a subject.

According to one aspect of the invention, an intraocular pressure (IOP) tonometer is provided that includes a contact lens configured to be located and retained on a user's eye, and a corneal sensor comprising a circular trace located proximate an outer peripheral edge of the contact lens. The circular trace surrounds an unobstructed area at a center region of the contact lens. The corneal sensor is configured to produce a detectable shift in resonance frequency in response to a change in IOP of the user's eye. The detectable shift in resonance frequency is wirelessly delectable by a receiving antenna.

According to another aspect of the invention, a method is provided for fabrication of an IOP tonometer of the present disclosure. The method includes depositing one or more layers of the circular trace onto a temporary substrate, removing the circular trace from the temporary substrate, and securing the circular trace onto an outer surface of the contact lens.

According to another aspect of the invention, a system is provided for monitoring intraocular pressure (IOP) of the user. The system includes an IOP tonometer as disclosed herein, a wearable device comprising the receiving antenna configured to encircle the circular trace of the IOP tonometer while the IOP tonometer is worn on the user's eye, and a data acquisition unit configured to receive the detected shift in resonance frequency from the wearable device and store the detected shift in resonance frequency. The receiving antenna is configured to wirelessly detect the shift in resonance frequency of the corneal sensor corresponding to a change in IOP of the user's eye.

According to another aspect of the invention, a method is provided for monitoring the intraocular pressure (IOP) of the user with a system of the present disclosure. The method includes placing the IOP tonometer on the user's eye, locating the wearable device on the user's face such that the receiving antenna encircles the circular trace of the IOP tonometer, sensing the shift in resonance frequency of the IOP tonometer in response to the change in IOP of the user's eye, wirelessly detecting the shift in resonance frequency of the IOP tonometer with the receiving antenna of the wearable device, transmitting the detected shift in resonance frequency from the wearable device to the data acquisition unit and storing the detected shift in resonance frequency on data storage media thereof, and analyzing the detected shift in resonance frequency to determine the change in IOP of the user.

Technical effects of IOP tonometers, systems, and methods as described above may in some arrangements include the ability to monitor the IOP of a subject over an extended period of time, in some cases, while the subject is asleep. Other aspects and advantages of this invention will be appreciated from the following detailed description.

### BRIEF DESCRIPTION OF THE DRAWINGS

FIG. 1A schematically represents a system for monitoring of intraocular pressure (IOP) in a subject both while awake and asleep in accordance with certain nonlimiting aspects of the invention. FIG. 1B schematically represents a series resistor-inductor-capacitor (RLC) resonant circuit of an IOP tonometer of the system of FIG. 1A and receiving antenna (reader coil) of the system of FIG. 1A.

FIGS. 2A through 2C represent an eye-wearable IOP tonometer of the system of FIG. 1A. FIG. 2A represents the IOP tonometer. FIG. 2B represents the IOP tonometer on the corneal anterior surface of an enucleated porcine eye. FIG. 2C schematically represents an exploded view of a stack of layers of a corneal sensor of the IOP tonometer.

FIGS. 3A through 3C schematically represent steps in a method of fabricating a IOP tonometer configured as shown in FIGS. 2A through 2C in accordance with aspects of the present invention. FIG. 3A represents a series of printing steps performed with an experimental setup comprising an automated nozzle injection system and a computer-controlled translation stage to fabricate corneal sensors on temporary removable substrates. FIG. 3B represents oxidative polymerization of PDA from dopamine hydrochloride where an in-situ monomer creation occurs during the reaction mechanism. FIG. 3C represents transfer and bonding of the as-printed corneal sensor onto a contact lens.

FIG. 4 represents experimental results of IOP tonometers configured as shown in FIGS. 2A through 2C and fabricated with an experimental setup as represented in FIG. 3A. In particular, FIG. 4 presents experimental measurement results for the storage and loss moduli (left graph) and the viscosity (right graph) of different ink formulations used in the fabrication of the experimental IOP tonometers.

FIGS. 5A through 5D represent results from various experimental investigations performed on IOP tonometers fabricated with an experimental setup as represented in FIG. 3A in accordance with aspects of the present invention. FIG. 5A represents modulus of the IOP tonometer, a bare control soft contact lens, and the corneal sensor alone. FIG. 5B represents modulus of the materials used in the stack of layers of the corneal sensor. FIG. 5C represents capacitance change of various silicone elastomer dielectrics. FIG. 5D represents electrical properties of an AgSEBS conductor in response to mechanical strain.

FIG. 6 represents an image of an experimental receiving antenna for use in the system of FIG. 1A in accordance with aspects of the present invention.

FIGS. 7A through 7D represent images (FIGS. 7A and 7B) and the corresponding FEA results (FIGS. 7C and 7D) of an IOP tonometer in accordance with aspects of the present invention under four different loading conditions, namely: flipping (FIG. 7A), folding (FIG. 7B), stretching up to 40% (FIG. 7C), and expanding up to 10% (FIG. 7D).

FIGS. 8A through 8F represent aspects of an experimental setup in accordance with the system of FIG. 1A in accordance with aspects of the present invention and results obtained therefrom. FIG. 8A schematically represents in-situ wireless IOP sensing system in accordance with aspects of the present invention. The IOP tonometer was tested in-vitro on freshly enucleated porcine eyes due to their similar size to the human eyes. FIG. 8B shows the external receiving antenna (reader coil) aligned over the IOP tonometer along the same axis. FIG. 8C represents the reflection spectra wirelessly collected from the IOP tonometer worn by the porcine eyes. The resonance frequency value of the IOP tonometer shifted downwards as the IOP increased because the capacitance and inductance of the LC resonator increased. The frequency response was almost linear in the physiologically relevant IOP range of 7.5-35 mmHg with responsivity of 29.1 kHz/mmHg, which corresponded to the sensitivity of 122.1 ppm/mmHg. FIGS. 8E and 8F show test results of the effect of the distance and the angle between the receiving antenna and the sensor on the sensing performance, respectively. The data showed that the distance until 10 mm and the angle until 50° didn't alter the initial resonance frequency. These results suggest that the maximal reading distance was about 10 mm and eye movements would not produce significant errors in pressure reading despite a reduction in the signal strength.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of what is depicted in the drawings, including the embodiment(s) depicted in the drawings and/or to which the drawings relate. The following detailed description also describes certain investigations relating to the embodiment(s) depicted in the drawings, and identifies certain but not all alternatives of the embodiment(s). As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

Disclosed herein are devices (referred to herein as tonometers), systems and methods that in preferred configurations are capable of continuous, unobtrusive, non-invasive 24-hour monitoring and recording of intraocular pressure (IOP) in subjects. Such recordings may be used to determine mean IOP more accurately than current daytime in-office measurements. As such, data obtained from these devices, systems and methods have the potential for significantly promoting the successful diagnosis, monitoring, and treatment of glaucoma.

The devices, systems and methods utilize an eye-wearable IOP tonometer that includes a wirelessly-addressable, ultra-thin, deformable corneal sensor anchored to an extended wear contact lens that can safely and comfortably interface with the corneal surface of human eyes. As used herein, the phrase “extended wear” indicates that the contact lens may be worn by a subject for extended periods of time (e.g., 24 hours or more) and may be worn both while awake and while sleeping with little or no negative side effects. The IOP tonometer may be wirelessly and functionally coupled to a receiving antenna for transmitting signals therebetween corresponding to IOP measurements. In some cases, the receiving antenna may be embedded inside a wearable device, which may be, as nonlimiting examples, a periocular device, eyeglasses, a skin-mountable facial patch (e.g., while the subject is awake), a sleeping mask (e.g., while the subject is asleep).

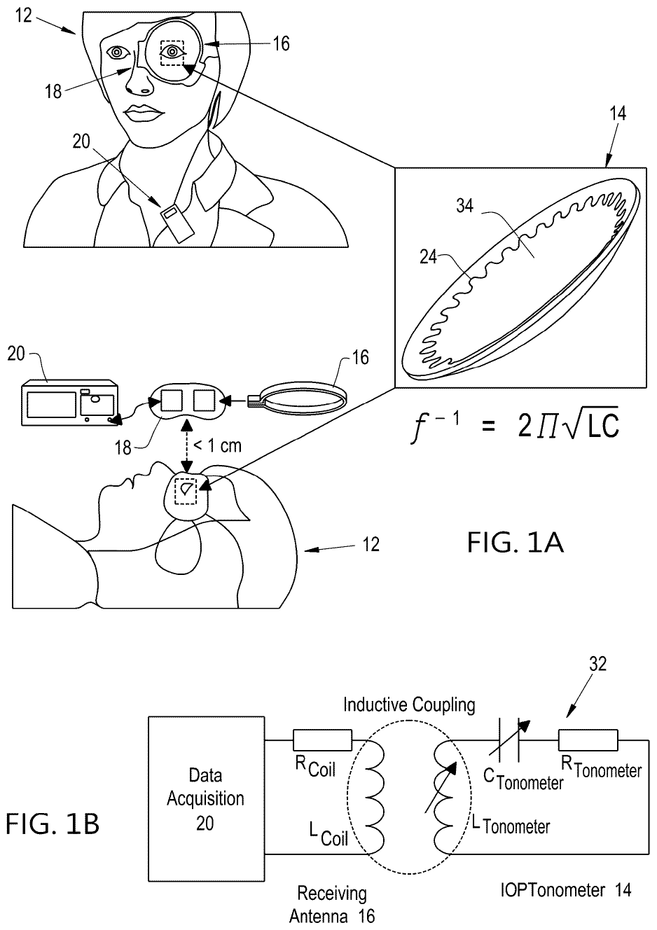

FIG. 1A illustrates a nonlimiting embodiment of a system for monitoring IOP of a user 12 using an IOP tonometer 14, a periocular device 18, and a data acquisition unit 20. The system allows 24-hour monitoring of IOP of a subject (user) 12, such as a glaucoma patient, while awake (top panel) and while sleeping (bottom panel). The subject 12 may wear the IOP tonometer 14 in the subject's eye in a manner consistent with existing extended wear contact lenses, such as those available for vision correction. During use, the IOP tonometer 14 may be used to continuously sense changes in IOP of the subject 12 and wirelessly transmit signals corresponding to such changes to a receiving antenna (reader coil) 16 of the periocular device 18. FIG. 1A schematically represents the IOP tonometer 14 as comprising a corneal sensor 24 formed on a contact lens 34. Nonlimiting examples of the periocular device 18 include a skin patch represented in the top panel of FIG. 1A and a sleeping mask represented in the bottom panel of FIG. 1A. As discussed in more detail below, the tonometer 14 comprises a series resistor-inductor-capacitor (RLC) resonant circuit 32, to which the receiving antenna 16 of the periocular device 18 is inductively coupled as schematically represented in FIG. 1B.

The periocular device 18 may communicate (wirelessly or via a wired connection) the received measurements to the data acquisition unit 20, which preferably includes a data storage device for recording and storing the measurements. The data acquisition unit 20 may be a portable or stationary electronic device (e.g., network analyzer, computer, mobile phone, tablet computer, or other electronic device with data storage capabilities). For portable embodiments, the data acquisition unit 20 may include a battery. In some cases, the data acquisition unit 20 may transmit the stored measurements to an external and/or remote computing system (e.g., smart phones, tablet computer, desktop computer, server, etc.) via a wireless connection (e.g., Bluetooth®) for storage, remote access, and/or analysis. In some cases, data comprising the measurements and/or analysis data relating thereto may be stored and remotely accessible from the remote computing system using a computer software application, an internet connection, and/or a wireless connection.

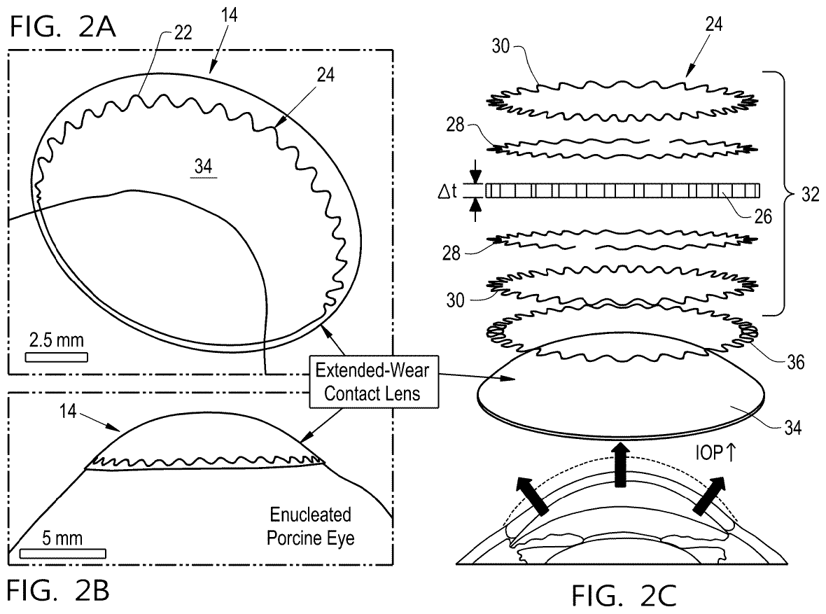

FIGS. 2A, 2B, and 2C represent a nonlimiting embodiment of the IOP tonometer 14 that includes a stack of layers configured into a narrow, stretchable serpentine coil that defines a circular trace 22 of the corneal sensor 24 (e.g., 250 μm-wide×66 mm-long×less than 10 μm-thick). The stack of layers preferably exclusively includes biocompatible, soft elastomers. For example, in FIG. 2C the stack of layers includes a highly deformable dielectric layer 26 (e.g., an ultra-soft silicone elastomer such as Silbione RT Gel 4717 A/B available from Bluestar Silicone, USA; Mechanical modulus (E)=5 kPa), a pair of inductive sending antenna layers 28 (e.g., elastomeric polystyrene-b-poly(ethylene-ran-butadiene)-b-polystyrene (SEBS) with Ag flakes) (AgSEBS), and a pair of encapsulation layers 30 (e.g., polydimethylsiloxane (PDMS)). The stack of layers 26, 28, and 30 defines the RLC resonant circuit 32 of the IOP tonometer 14. The RLC resonator circuit 32 may be secured to an extended wear contact lens 34 with an adhesive layer 36 (e.g., polydopamine (PDA)).

An increase of IOP may be wirelessly measured by wirelessly detecting changes in the RLC resonator 32. Specifically, an increase in IOP results in a thinning of the exceptionally soft dielectric layer 26 and a lateral stretching of the inductive sending antenna layers 28 of the corneal sensor 24. This in turn results in an increase in capacitance and inductance of the inductive sending antenna layers 28 that produces a detectable shift of the resonance frequency (∫⁻¹=2π\sqrt{LC}) , where L and C are the inductance and capacitance of the sending antenna layers 28, respectively. This shift in resonance frequency is detectable by the receiving antenna 16. The sensitivity of this type of variable LC circuit-based tonometer may be between 70 to 200 ppm mmHg⁻¹, which is sufficiently large for the detection of small changes in IOP.

For wireless transmission of signals and power, the embedded receiving antenna 16 of the periocular device 18 may be electromagnetically coupled to the sending antenna layers 28 in the corneal sensor 24. During the measurement process, the distance between the sending antenna layers 28 and the receiving antenna 16 may be maintained within about 30 mm, and more preferably within about 10 mm.

In some embodiments, the stack of layers of the corneal sensor 24 may be at least 10-fold thinner than regular commercial contact lens 34 (e.g., less than 10 μm for the corneal sensor vs. about 0.08 to 0.18 mm for the contact lens), such that the corneal sensor 24 does not substantially increase the overall thickness of the IOP tonometer 14. The corneal sensor 24 may also be 10-fold softer and more stretchable that regular commercial contact lens 34. These properties allow the IOP tonometer 14 to interface intimately across the corneal surface of human eyes, which promotes patient comfort and superior sensitivity (e.g., greater than 150 ppm mmHg⁻¹). In certain cases, the corneal sensor 24 may be located proximate, e.g., on or near, a peripheral edge of the contact lens 34 at a diameter significantly larger than a physiological pupil, with a ring-shaped configuration that will account for greater than 10% of the total surface area of the contact lens to reduce interference with the wearer's vision, such that the circular trace 22 surrounds an unobstructed area at a center region of the contact lens.

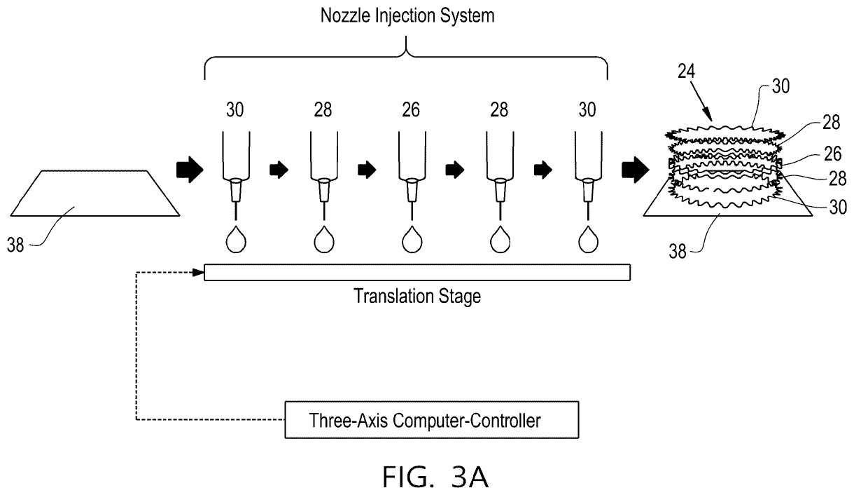

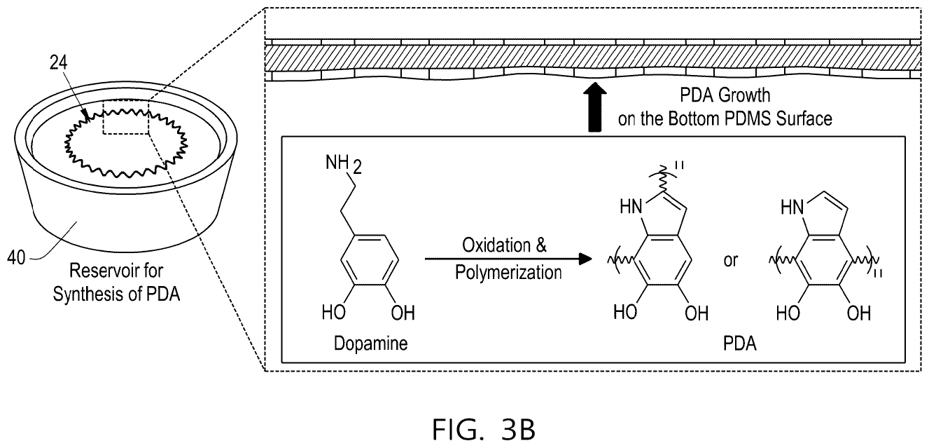

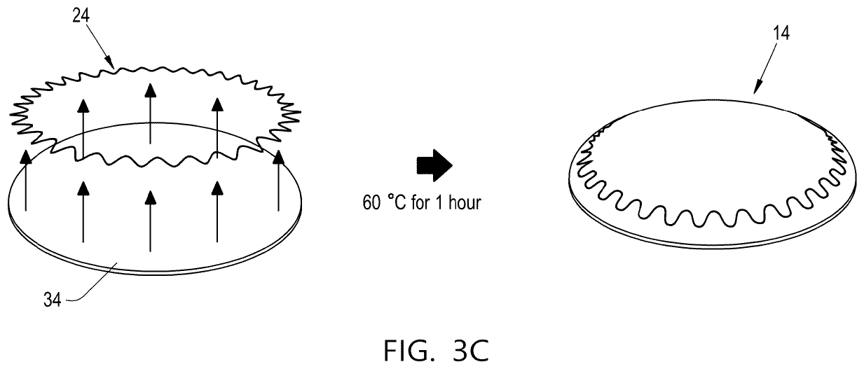

Various methods may be used to fabricate the IOP tonometer 14. An exemplary, nonlimiting method is represented in FIGS. 3A, 3B, and 3C. FIG. 3A schematically represents an experimental setup that was used to fabricate corneal sensors 24 configured as shown in FIGS. 2A through 2C. The experimental setup was adapted to perform a direct-ink-writing process for sequentially writing (i.e., depositing) the stack of layers 26 through 30 of the corneal sensors 24 on temporary removable substrates 38. FIG. 3A schematically represents the experimental setup as including an automated nozzle injection system and a three-axis computer-controlled translation stage. The three-axis computer-controlled translation stage was a Nordson EFD having a resolution of 1 μm and repeatability of +/−3 μm. Preferred methods are capable of writing multiple layers of linear and curvilinear traces uniformly at the microscale (e.g., greater than 100 μm in width and greater than 10 μm in thickness) in a series of pre-programmed steps, enabling for rapid prototyping (e.g., greater than 10 units per print) at a printing speed of, for example, about 4 mmₛ⁻¹.

Rheological properties of the materials used for fabricating the stack of layers 26 through 30 may be adjusted by selectively mixing silica particles into the compositions thereof in predetermined amounts. For example, the silica particles may be added in an amount sufficient to adjust shear-thinning flow behavior of the materials to promote the capability of being dispensable through a nozzle with high fidelity. Specifically, the weight percentage of silica particles may be adjusted to control the viscosity and shear moduli of the materials. In this manner, the materials may be adjusted to control their “printability” in terms of a ratio of loss modulus (G″) to storage modulus (G′).

Once the corneal sensor 24 has been produced, the stack of layers 26 through 30 may be integrated (e.g., anchored) onto an outer surface of a soft contact lens 34, for example, with the adhesive layer 36. In some cases, the corneal sensor 24 may be secured to a commercially available contact lens 34. Examples of suitable methods for securing the corneal sensor 24 to the contact lens 34 include various wet chemical anchoring processes. A particular but nonlimiting processes adheres the corneal sensor 24 to the contact lens 34 with polydopamine (PDA) which benefits from PDA's relatively straightforward synthesis, biocompatibility, and strong adhesive properties in both dry and wet conditions.

As represented in FIG. 3B, the corneal sensor 24 may be located in a reaction bath 40 that includes a precursor to PDA, such as dopamine hydrochloride in a slightly basic (pH=8.5) buffered solution. The PDA can be polymerized through an oxidative polymerization process wherein oxygen present in the atmosphere causes the dopamine hydrochloride to first cyclize and then polymerize to form the polydopamine. Importantly, the slow diffusion of oxygen into the reaction bath 40 allows for a controlled polymerization that produces a conformal coating on any exposed surfaces. As such, the growth of the PDA may be limited to specific surfaces of the corneal sensor 24, such as a single side of the stack of layers 26 through 30 (e.g., a bottom intended to contact the contact lens) as represented in the enlarged window of FIG. 3B.

Alternatively, or in addition, the adhesive layer 36 (e.g., PDA) may be formed on the contact lens 34. In such cases, the controlled polymerization described above allows for selective templating of the surface of the contact lens 34. In this way, the adhesive layer 36 can be formed only in specific regions of interest. Therefore, the adhesive layer 36 is not required to be coated across an entire area of the contact lens 34. As such, the contact lens 34 may be used without any perceived change in color (i.e., the light-yellow PDA will not be in the field of view of the wearer). Thus, placement of this biocompatible, electronically-insulating, strong adhesive layer 36 can be done in a spatially controlled manner.

Once the adhesive layer 36 is conformally-coated to the corneal sensor 24 and/or the contact lens 34, the corneal sensor 24 can be easily adhered to the contact lens 34 via mild heating of the adhesive layer 36 for a brief period of time to yield a final product (IOP tonometer 14) as represented in FIG. 3C.

Once completed, the IOP tonometer 14 is capable of undergoing significant mechanical and chemical stresses without the loss of adhesion between the contact lens 34 and the corneal sensor 24 due to the strong intermolecular interactions associated with the catechol moieties of the PDA in the adhesive layer 36. The adhesive properties of the adhesion layer 36 may be adjusted by controlling polymerization time, polymer temperature, and oxygen content of the polymerization solution.

To avoid or reduce the likelihood of surface discontinuity, the circular trace 22 of the corneal sensor 24 may be adjusted such that it can be stretched effectively to adopt the interfacial stress when contacted to the curvilinear surface of the contact lens 34, and when the contact lens further alter their shape when ultimately placed on an eye.

The completed IOP tonometer 14 may be thoroughly rinsed with, for example, a sterile saline solution, followed by additional sterilization with a cleaning and disinfecting care solution containing formulated hydrogen peroxide (H₂O₂) to remove any residual proteins. The IOP tonometer 14 may be stored in a conventional contact lens solution and case.

The wet chemical anchoring process preferably does not alter the shape or conformability of the contact lens 34, nor affect their intrinsic wettability. The adhesion layer 36 may be configured to provide mechanical and chemical stability against lens handling, fitting, cleaning, and disinfecting processes. In some cases, the IOP tonometer 14 may be configured to be disposable after one or more uses.

For embodiments in which the corneal sensor 24 is anchored onto a commercially available contact lens 34, it is believed that the wet chemical anchoring process may allow for integration without substantially altering the intrinsic properties of the contact lens, for example, in terms of biocompatibility, softness, wettability, oxygen transmissibility, transparency, and ergonomic curvature. Suitable contact lenses 34 may provide excellent biocompatibility, softness (e.g., E=0.2-2 MPa), transparency (e.g., greater than 90%), oxygen transmissibility (e.g., 10-200 Dk/t), wettability (e.g., water content=30-80%), and ergonomic curvature (e.g., 8.3-9.0 mm).

Nonlimiting embodiments of the invention will now be described in reference to experimental investigations leading up to the invention.

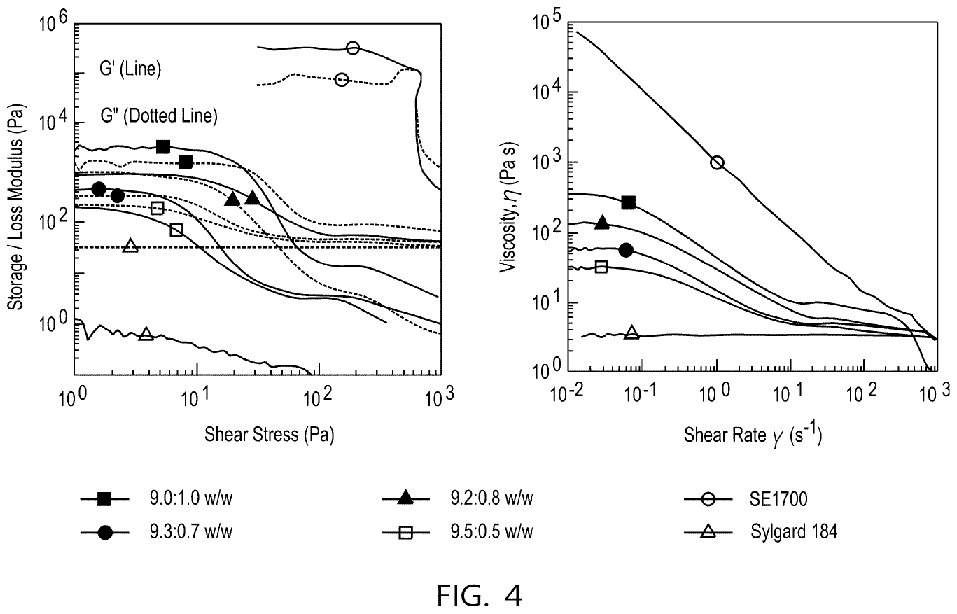

FIG. 4 represents measurements of the storage and loss moduli (left graph) and the viscosity (right graph) of differently-formulated PDMS inks with different weight ratios of a base resin, dilute resin, and SiO₂-PS particles ranging from 9.0:1.0 to 9.3:0.7, 9.2:0.8, and 9.5:0.5, as compared to commercial control groups of a dispensable silicone ink (SE 1700, Dow Corning) and a bare PDMS ink (Sylgard 184, Dow Corning). The results exhibited a gel-like viscoelastic behavior (i.e., G′ greater than G″) within the linear viscoelastic (LVE) region (plateau regions) that indicates a printable range without distorting structural integrity. On the other hand, an ink with 9.5:0.5 ratio and the Sylgard 184 ink exhibited a liquid-like viscoelastic behavior due to their dominating G″ at all shear stresses, which thereby precluded their use for dispensing through a nozzle. These inks exhibited the shear-thinning behavior in which their viscosity decreased with an increase of the shear rate, whereas the Sylgard 184 ink exhibited a zero-shear-rate behavior (plateau curve) at all shear rates.

FIGS. 2A and 2B provide images of the corneal sensor 24 built on a commercial extended wear contact lens 34 (Air Optix Night & Day Aqua, Alcon). Investigations revealed that the ergonomic design of the commercial contact lens 34 enabled the formation of a seamless contact to the corneal anterior surface of an enucleated porcine eye, which provides general anatomical similarity to the human eye.

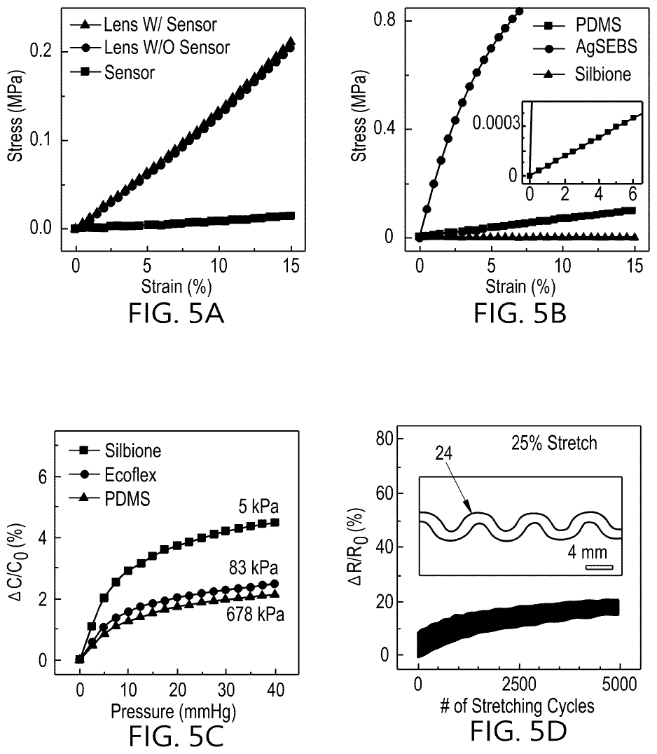

FIGS. 5A through 5D represent results from various experimental investigations performed on IOP tonometers 14 fabricated with the experimental setup represented in FIG. 3A. FIG. 5A represents that the IOP tonometer had a similar modulus (E=1.408±0.129 MPa) to a bare control soft contact lens (1.367±0.120 MPa, Air Optix Night&Day Aqua, Alcon) by virtue of the low effective modulus of the bare printed corneal sensor (0.101 ±0.008 MPa) and its small area occupying only about 10% of the total surface area of the contact lens. FIG. 5B represents that the modulus of Silbione dielectric (5.0 kPa±1.3 kPa) was much smaller than those of AgSEBS (14±1.9 MPa) and PDMS (0.68±0.09 MPa), which allows for high sensitivity of the RLC resonator 32 by changing thickness of the dielectric easily when the IOP is applied. For example, FIG. 5C represents capacitance change of various silicone elastomer dielectrics including Silbione, Ecoflex (E=83±9.3 kPa), and PDMS under human IOP ranges. The RLC resonator 32 employing the Silbione dielectric with the smallest modulus provided the highest response. In FIG. 5D, electrical properties of AgSEBS conductor in response to mechanical strain were investigated for reliable operation of the RLC resonator 32 on the contact lens 34. Resistance of AgSEBS increased only about 15% (AR/R₀) after 5,000 cycles of stretching (25% tensile strain) and relaxation.

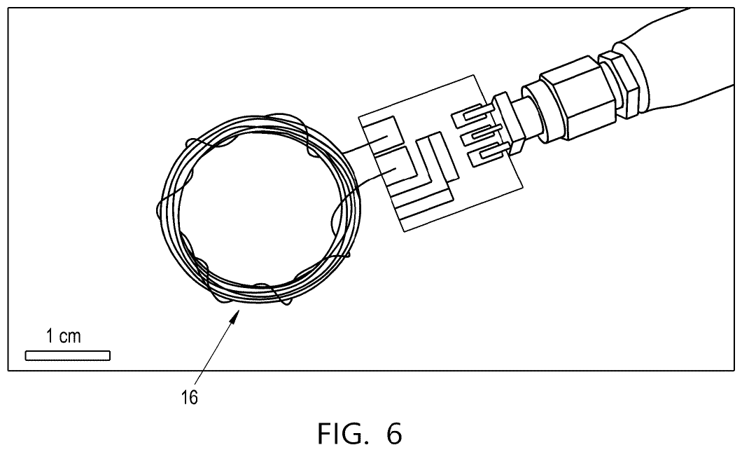

FIG. 6 represents an experimental representation of the receiving antenna 16 using a wound, enamel-covered copper coil that was tuned to approximately 240 MHZ with outer and inner diameters of 25 and 20 mm, respectively. The experimental receiving antenna 16 was connected to a data acquisition unit 20 via a USB cable to provide time-varying magnetic fields at approximately 15-25 G. Measurements obtained with the experimental receiving antenna 16 indicated a resulting resistance and inductance are 3.5-4.0 22 and 450-480 μH, respectively.

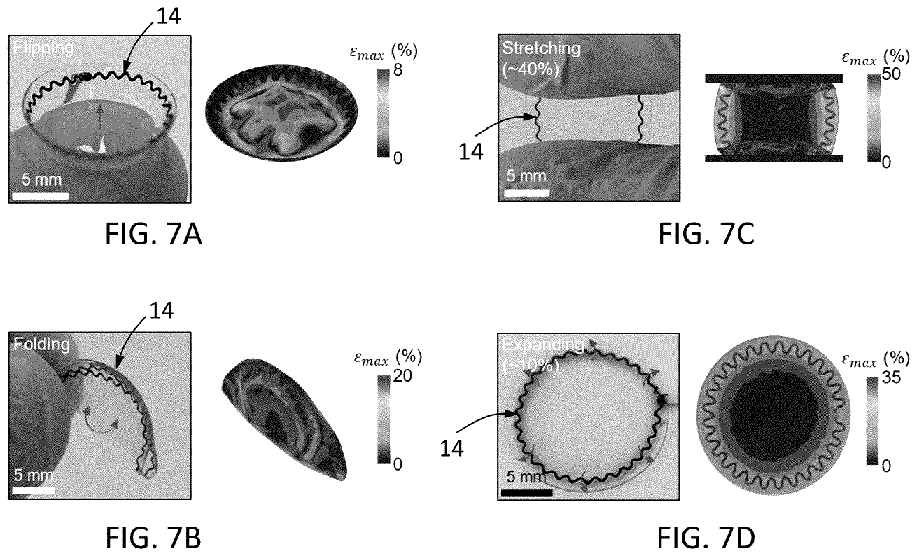

FIGS. 7A through 7D summarize results of experimental and computational (FEA) modeling showing maximum principle strain (Emax) of the IOP tonometer 14 under four different loading conditions including (FIG. 7A) flipping, (FIG. 7B) folding, (FIG. 7C) stretching (up to 40%), and (FIG. 7D) expanding (up to 10%). These results showed that the maximum principle strain (Emax) of the IOP tonometer 14 remained lower than about 10% under these loading conditions. For example, when completely flipped over, the IOP tonometer 14 experienced little deformation with a maximum strain of less than 1%. When folded in half along the symmetric axis, the maximum strain (less than 10%) was concentrated at the folding line of the device. When stretched uniaxially and expanded uniformly, the results consistently showed maximum strain of no more than 10%. Therefore, these results suggested that the stack of layers 26 through 30 of the corneal sensor 24 experienced little deformation under these high loading conditions; far below the fracture limit of the materials of the stack of layers (greater than about 75%). These findings also implied that the effect of the corneal sensor 24 on the intrinsic mechanical properties of the commercial contact lens 34 was insubstantial.

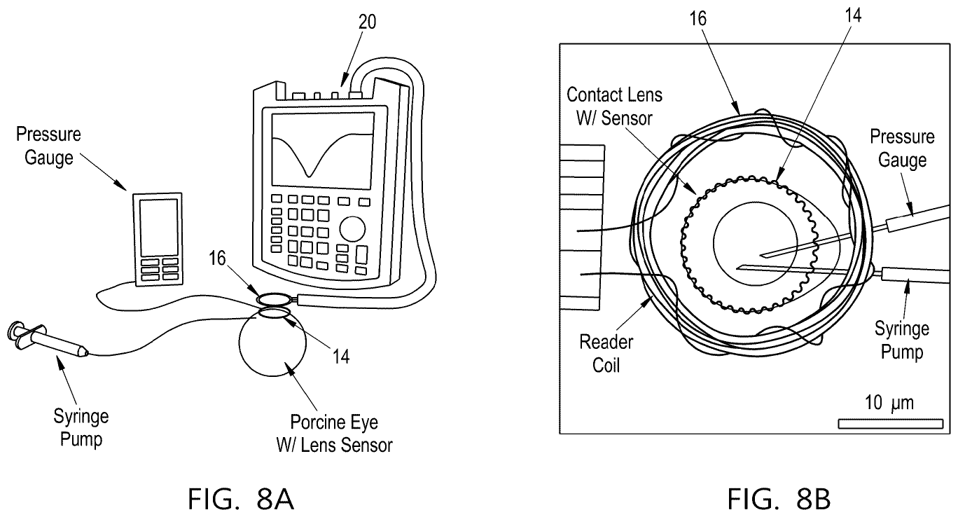

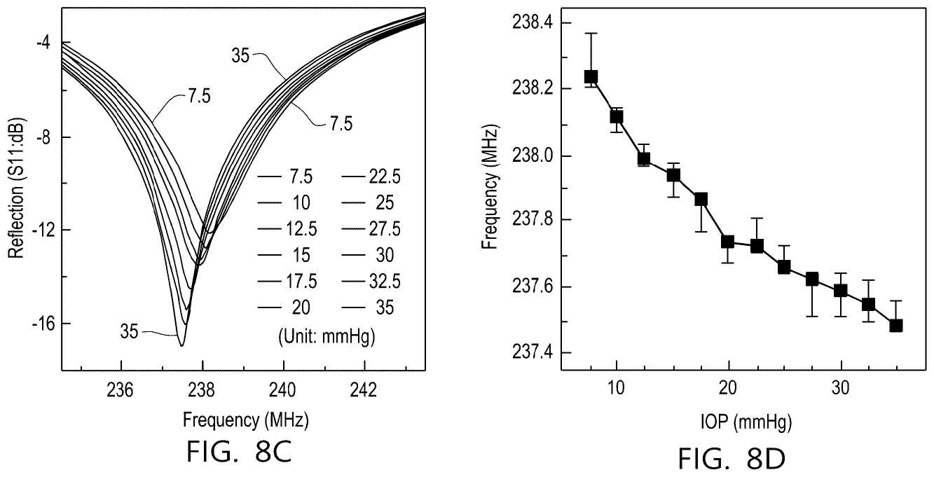

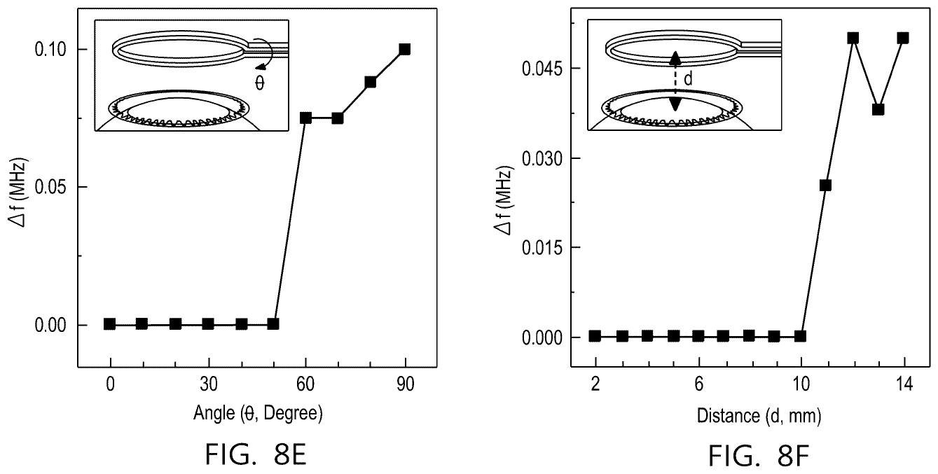

FIGS. 8A through 8F relate to characterization of ex vivo investigations of wireless performance of the system under various conditions using porcine eyes. FIG. 8A schematically represents an experimental setup used for these investigations. The IOP tonometer 14 was located on the corneal surface of an enucleated porcine eye under a receiving antenna 16 (distance=1 cm) that was connected to a portable data acquisition unit 20 with a calibrated frequency range of 230-250 MHZ. The porcine eye was cannulated with two needles. The first needle was connected to a syringe pump to modulate the IOP by infusing a saline solution into the anterior chamber of the porcine eye. The second needle was connected to a pressure gauge to simultaneously measure the IOP for calibration. Prior to each measurement, the IOP of the porcine eye was stabilized at 7.5 mmHg. Five measurements were taken at each given IOP interval, which were saved and exported from the data acquisition unit 20 for post-data analysis. The saline infusion was cut after the IOP reached 35 mmHg (maximum value), which allowed the IOP to decrease back to the equilibrium pressure (7.5 mmHg). The results revealed that the IOP increased at intervals of 2.5 mmHg (beginning at 7.5 mmHg and ending at 35 mmHg) with the measured responsivity or sensitivity of 29.1 kHz mmHg⁻¹ (correspondingly, 122.1 ppm mmHg⁻¹), which are within a comparable range (70-200 ppm mmHg⁻¹) of conventional ocular tonometers.

While the invention has been described in terms of specific or particular embodiments and investigations, it should be apparent that alternatives could be adopted by one skilled in the art. For example, the system and its components could differ in appearance and construction from the embodiments described herein and shown in the figures, functions of certain components of the system could be performed by components of different construction but capable of a similar (though not necessarily equivalent) function, and appropriate materials could be substituted for those noted. Accordingly, it should be understood that the invention is not necessarily limited to any embodiment described herein. It should also be understood that the phraseology and terminology employed above are for the purpose of describing the disclosed embodiments and investigations, and do not necessarily serve as limitations to the scope of the invention.

1. An intraocular pressure (IOP) tonometer comprising:
a contact lens configured to be located and retained on a user's eye; and
a corneal sensor comprising a circular trace located proximate an outer peripheral edge of the contact lens, wherein the circular trace surrounds an unobstructed area at a center region of the contact lens,
wherein the corneal sensor is configured to produce a detectable shift in resonance frequency in response to a change in IOP of the user's eye, and
wherein the detectable shift in resonance frequency is wirelessly delectable by a receiving antenna.

2. The IOP tonometer of claim 1, wherein the circular trace defines a circular serpentine pattern.

3. The IOP tonometer of claim 1, wherein the circular trace comprises a dielectric layer, a pair of inductive sending antenna layers, and a pair of encapsulation layers that define an inductor-resonance (LC) resonator.

4. The IOP tonometer of claim 3, wherein the delectable shift in resonance frequency is due to a thinning of the dielectric layer and a lateral stretching of the pair of inductive sending antenna layers that increase capacitance and inductance of the inductive sending antenna layers.

5. The IOP tonometer of claim 3, wherein the dielectric layer comprises a silicone elastomer dielectric, the inductive sending antennas comprise AgSEBS, and the encapsulation layers comprise PDMS.

6. The IOP tonometer of claim 1, further comprising an adhesive layer anchoring the circular trace to the contact lens.

7. The IOP tonometer of claim 6, wherein the adhesive layer comprises PDA.

8. A method of fabricating the IOP tonometer of claim 1, the method comprising:
depositing one or more layers of the circular trace onto a temporary substrate;
removing the circular trace from the temporary substrate; and
securing the circular trace onto an outer surface of the contact lens.

9. The method of claim 8, further comprising:
forming an adhesive material on the circular trace,
wherein the securing step comprises securing the circular trace to the outer surface of the contact lens with the adhesive material.

10. The method of claim 8, further comprising:
forming an adhesive material on the contact lens,
wherein the securing step comprises securing the circular trace to the outer surface of the contact lens with the adhesive material.

11. The method of claim 10, wherein the adhesive material is formed proximate the outer peripheral edge of the contact lens and does not overlay the unobstructed area at the center region of the contact lens.

12. The method of claim 8, wherein the contact lens is a commercially available contact lens.

13. A system for monitoring intraocular pressure (IOP) of a user, the system comprising:
the IOP tonometer of claim 1;
a wearable device comprising the receiving antenna configured to encircle the circular trace of the IOP tonometer while the IOP tonometer is worn on the user's eye, wherein the receiving antenna is configured to wirelessly detect the shift in resonance frequency of the corneal sensor corresponding to a change in IOP of the user's eye; and
a data acquisition unit configured to receive the detected shift in resonance frequency from the wearable device and store the detected shift in resonance frequency.

14. The system of claim 13, wherein the wearable device comprises at least one of a periocular device, eyeglasses, a skin-mountable facial patch, and a sleeping mask.

15. The system of claim 13, wherein the wearable device comprises at least one of a sleep mask and a circular patch configured to be releasably secured to the user's face.

16. A method of monitoring the intraocular pressure (IOP) of a user with the system of claims 13, the method comprising:
placing the IOP tonometer on the user's eye;
locating the wearable device on the user's face such that the receiving antenna encircles the circular trace of the IOP tonometer;
sensing the shift in resonance frequency of the IOP tonometer in response to the change in IOP of the user's eye;
wirelessly detecting the shift in resonance frequency of the IOP tonometer with the receiving antenna of the wearable device;
transmitting the detected shift in resonance frequency from the wearable device to the data acquisition unit and storing the detected shift in resonance frequency on data storage media thereof; and
analyzing the detected shift in resonance frequency to determine the change in IOP of the user.

17. The method of claim 16, wherein the circular trace comprises a dielectric layer, a pair of inductive sending antenna layers, and a pair of encapsulation layers that define an inductor-resonance (LC) resonator, and wherein the shift in resonance frequency is due to a thinning of the dielectric layer and a lateral stretching of the pair of inductive sending antenna layers that increase capacitance and inductance of the inductive sending antenna layers.

18. The method of claim 16, wherein the circular trace is electromagnetically coupled to the wearable device in a manner sufficient to provide for wireless transmission of signals and power therebetween.

19. The method of claim 16, further comprising:
transmitting the determined change in IOP from the data acquisition unit to a remote computing system; and
providing remote access to the determined change in IOP and/or analysis data relating thereto with the remote computing system.

20. The method of claim 16, wherein the user's eye is continuously monitored for the change in IOP for at least 24 hours, and wherein the user is asleep for at least some of the at least 24 hours.
